(19) United States
(12) Patent Application Publication
(10) Pub. No.: US 2026/0083892 A1
(43) Pub. Date: Mar. 26, 2026

(54) MUCUS EXTRACTION DEVICE AND METHOD OF USE

(71) Applicant: Playhouse Method Inc., Los Angeles, CA (US)

(72) Inventors: Sydney Wiseman, Los Angeles, CA (US); Louis Séguin, Montreal (CA); Matthew Callahan, New York, NY (US); Juliette Alexia Laroche, New York, NY (US); Thomas Overthun, Saint Petersburg, FL (US)

(21) Appl. No.: 19/336,407

(22) Filed: Sep. 22, 2025

Related U.S. Application Data
(60) Provisional application No. 63/697,318, filed on Sep. 20, 2024.

(51) Int. Cl. A61M 1/00

(52) U.S. Cl. CPC A61M 1/682; A61M 2205/587; A61M 2205/59; A61M 2210/0618

(57) ABSTRACT

A mucus extraction device and method of use includes a compressible body that creates suction through a nasal tip, a housing including a lighting member supported therein, and a grip that is configured to be grasped by a user during operation of the mucus removal device. The mucus extraction device can be operated one-handed and the internal lighting member can be turned on to glow during use.

### CROSS-REFERENCE TO RELATED APPLICATIONS

This application claims the benefit of priority to U.S. Provisional Patent Application No. 63/697,318, filed Sep. 20, 2024 and entitled “Mucus Extraction Device and Method of Use” the entire contents of the application being incorporated herein by reference in its entirety.

### TECHNICAL FIELD

This disclosure relates generally to a mucus or fluid removal device, more particularly, to a manual device for mucus extraction including a compressible body that creates suction through a tube fluidly connected to the flexible body, and a housing that includes a lighting member and a grip that is configured to be grasped by a user during operation of the mucus removal device.

### BACKGROUND

When children are young and have nasal congestion, they often cannot remove the mucus (also known as snot) from their nose by blowing it out, as an adult might. Nasal congestion if not rectified can create discomfort in a child, which can result in crying, poor breathing, and lack of sleep, all of which can cause anxiety for parents. Nasal aspirators are a known device that can help parents extract the mucus from the nasal cavity of children to increase comfort and improve the quality of breathing for the child. There are traditionally two forms of nasal aspirators, manual nasal aspirators and electronic nasal aspirators.

Manual nasal aspirators, such as the exemplary prior art device shown in FIG. 1, generally include a distal end 110 having a suction tip 112 for insertion into the nose (or other orifice) of the child and a bulbous body 114 for squeezing to create the suction. In use, the suction tube is inserted within the nose of the child and an adult then squeezes the bulbous body to create suction to remove the mucus secretions, which are sucked into the suction tube. As will be appreciated, to function properly the body of the manual nasal aspirator that is used to create the suction must be of a sufficient size and be flexible so that it may be squeezed to create sufficient suction.

Electronic nasal aspirators, such as the exemplary prior art device shown in FIG. 2, are powered and do not require squeezing of a flexible body to create the suction. As with manual aspirators, electronic aspirators include a distal end 210 having suction tip 212 for insertion into the nose (or other orifice) and housing 216. The housing 216 in electronic aspirators houses the electronic components and is made of suitable rigid materials to safely contain the motor, battery and other mechanisms and is not bulbous, unlike the manual devices. The use of a rigid housing in electronic aspirators does not affect their functionality because, unlike manual nasal aspirators, the adult does not squeeze a bulbous portion to create suction, but rather suction is created by turning on the motor of the electronic aspirator. The mucus is then sucked from the child and into the housing 216, for example into a collection container 218. Some electronic aspirators include additional bells and whistles, for example, a beam of light, a rechargeable battery, and some even play music to soothe the child.

With either the manual or electronic versions, the use and outcomes are generally the same. An adult inserts the suction tip into a nostril and either turns on the device to create the suction or squeezes the body to create suction. Once suction is created the mucus (snot) moves into the suction tip or tube and may travel into a collection chamber. The device is then removed, and the process can be repeated in the other nostril.

### SUMMARY OF THE DISCLOSURE

Both manual and electronic aspirators are generally effective. However, electronic aspirators are often noisy, can be difficult to operate, and heavy to hold because they include a motor to supply power, and may include other components such as lights or sound. Manual aspirators are more simplistic, lighter weight, but the bulbous portion can be large and difficult to squeeze for adults with smaller hands and generally do not include many bells and whistles. Both types are functional but can create anxiety in the adult and child during use as they require insertion into the nostrils, or other cavities to suck out mucus.

Illustrative embodiments of the present disclosure provide an improved mucus suction instrument that includes a flexible body, a nasal tip that is supported by and fluidly connected to the body at one end and configured and dimensioned at the other end to sit outside of the nose adjacent the nostril or be configured to fit into the nostril of a child, and a housing also supported by the body that includes a light supported therein and a grip that is configured to be grasped by a user during operation of the mucus removal device.

The illustrative embodiment may also be designed with a whimsical overall design, such as that of an animal, an imaginary creature, or other fanciful design to entertain the child during use. In one embodiment the design is a narwhal, and the “horn” or “tusk” of the narwhal is the nasal tip. The nasal tip may be tapered from a base adjacent the body to an opening or distal end of the tip. In one embodiment, the design includes a “tail” or other extension that is sized as a grip for use during insertion.

In another illustrative embodiment, the flexible body is at least partially translucent and provides a soft glow by filtering the light that is emitted by the light supported in the housing when it is turned on.

In yet another illustrative embodiment the mucus extraction device comprises a compressible body including an interior cavity, a nasal tip in fluid communication with the compressible body, a housing including a grip member. The housing extending from the body. The device further includes a light assembly disposed within at least one of the interior cavity of the body and an opening formed within the housing, the lighting assembly including a light diffusing element; and wherein the grip member is configured to be grasped by a user during operation of the mucus extraction device and to maintain its shape when grasped.

In an illustrative embodiment, the mucus extraction device further comprises an attachment member for removably connecting the body to the housing of the mucus extraction device. In another illustrative embodiment, the attachment member includes the light diffusing element. In an additional illustrative embodiment, the compressible body is at least partially translucent. In another illustrative embodiment the grip is rigid.

In another illustrative embodiment, mucus extraction device comprising a compressible body including an interior cavity, the body being at least partially translucent; a nasal tip supported on a distal portion of the compressible body and in fluid communication therewith; a housing including a grip member, and a light supported within the interior cavity. The housing and grip member configured to be removably detachable from the body, and the grip member being disposed at a proximal end of the mucus extraction device; wherein the light is positioned to shine toward the nasal tip and to emit light through the at least partially translucent body.

In another illustrative embodiment, the grip member is configured to be grasped by a user during operation of the mucus extraction device. In yet another illustrative embodiment, the grip member is configured to maintain its shape when grasped. In an additional illustrative embodiment, the mucus extraction device further comprises an attachment member for removably connecting the body to the housing of the mucus extraction device. In an exemplary embodiment, the attachment member includes a light diffusing element. In another exemplary embodiment, the mucus extraction device further comprises a light assembly disposed within the interior cavity of the body, the lighting assembly including a light diffusing element.

These aspects of the disclosure are not meant to be exclusive and other features, aspects, and advantages of the present disclosure will be readily apparent to those of ordinary skill in the art when read in conjunction with the following description, appended claims, and accompanying drawings.

### BRIEF DESCRIPTION OF THE DRAWINGS

The foregoing features may be more fully understood from the following description of the drawings. Various aspects of at least one embodiment are discussed below with reference to the accompanying figures, which are not necessarily drawn to scale, emphasis instead being placed upon illustrating the principles disclosed herein. Since it is often impractical or impossible to illustrate and describe every possible embodiment, the provided figures depict one or more exemplary embodiments. The figures are incorporated in and constitute a part of this specification but are not intended as a definition of the limits of any embodiment. Accordingly, the figures are not intended to limit the scope of the invention. Like numbers in the figures denote like elements. For purposes of clarity, not every component may be labeled in every figure.

FIG. 1 is an exemplary prior art manual mucus extraction device;

FIG. 2 is an exemplary prior art electronic mucus extraction device;

FIGS. 3A-3B are perspective views of an exemplary embodiment of a manual mucus extraction device according to the present disclosure;

FIG. 4 is a partially exploded view of the embodiment of FIGS. 3A-3B including a body and a housing;

FIG. 5 is an exploded view of the embodiment of FIGS. 3A-3B;

FIG. 6 is a cross-sectional side view of the body and housing taken along lines 6-6 of FIGS. 3A-3B illustrating exemplary placing of internal components;

FIG. 7 is a rear, perspective view of the housing of FIG. 4, with a portion removed to show internal components;

FIG. 8 is a bottom perspective view of the housing of FIG. 7;

FIGS. 9A-9E are front, side, top, rear and second rear and front perspective views of the mucus extraction device of FIGS. 3A-3B being held and ready for use; and

FIG. 10 is a side perspective schematic view of the mucus extraction device of FIGS. 3A-3B being held and during use.

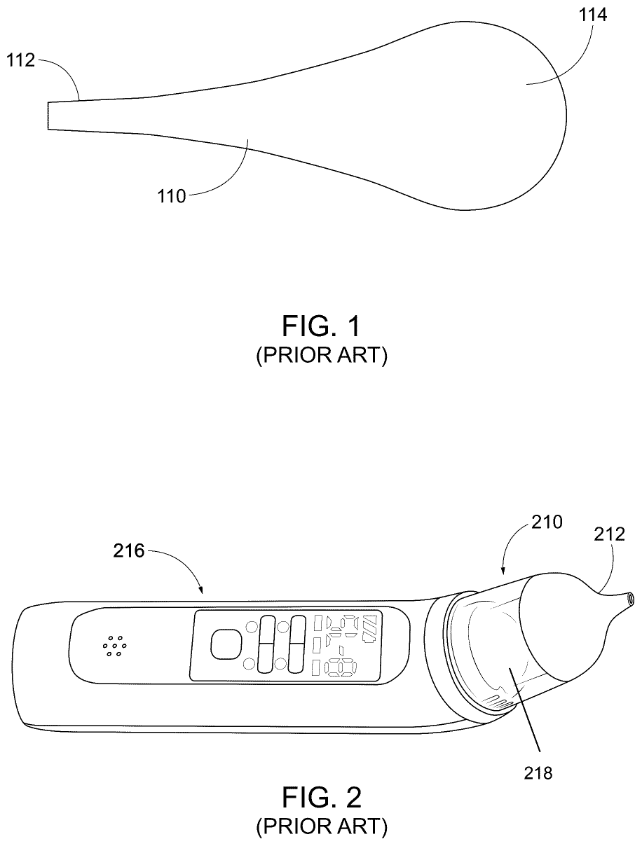

### DESCRIPTION OF THE ILLUSTRATIVE EMBODIMENTS

This disclosure includes examples and references of exemplary embodiments for the purposes of disclosure and is not intended to limit the scope of the devices, systems and methods disclosed herein. Exemplary embodiments of the present disclosure will be described in detail with reference to the accompanying drawings but are not limited in application to the details of construction and the arrangement of components set forth in the following description or illustrated in the accompanying drawings. Also, the phraseology and terminology used herein is for the purpose of description and should not be regarded as limiting. For example, the term “mucus” is used broadly to refer to a viscid slippery secretion that Smay originate from any part of the body and is not limited to nasal secretions. Any references to examples, embodiments, components, elements or acts of the device herein referred to in the singular may also embrace embodiments including a plurality, and any references in plural to any embodiment, component, element, or act herein may also embrace embodiments including only a singularity (and/or unitary structure). References in the singular or plural form or unitary or separate components are not intended to limit the presently disclosed device, its components, acts, elements or how they are connected unless expressly stated. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. It will be understood to one of skill in the art that the devices, methods and systems are capable of implementation in other embodiments and of being practiced or of being carried out in various ways.

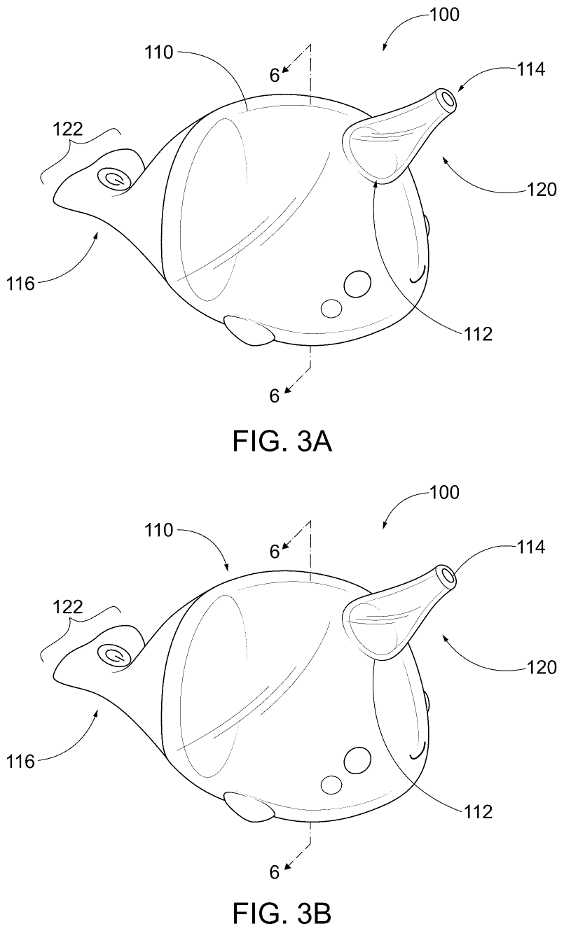

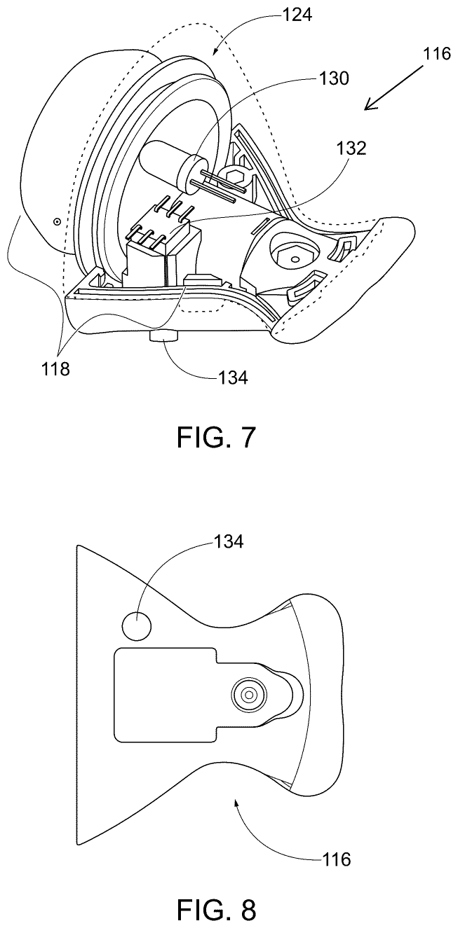

Referring initially to FIGS. 3A and 3B, the disclosed device provides an improved mucus extraction tool 100 that includes a flexible, i.e. compressible body 110, a nasal tip 120 that extends from and is supported by the body 110 and which may be configured and to sit outside of the nose adjacent the nostril or be configured and dimensioned for insertion into the nostril of a child. A housing 116 that includes a light assembly 118 (shown in FIG. 7) and a grip 122 that extends from the housing 116. The grip 122 is configured to be grasped by a user during operation of the mucus removal device.

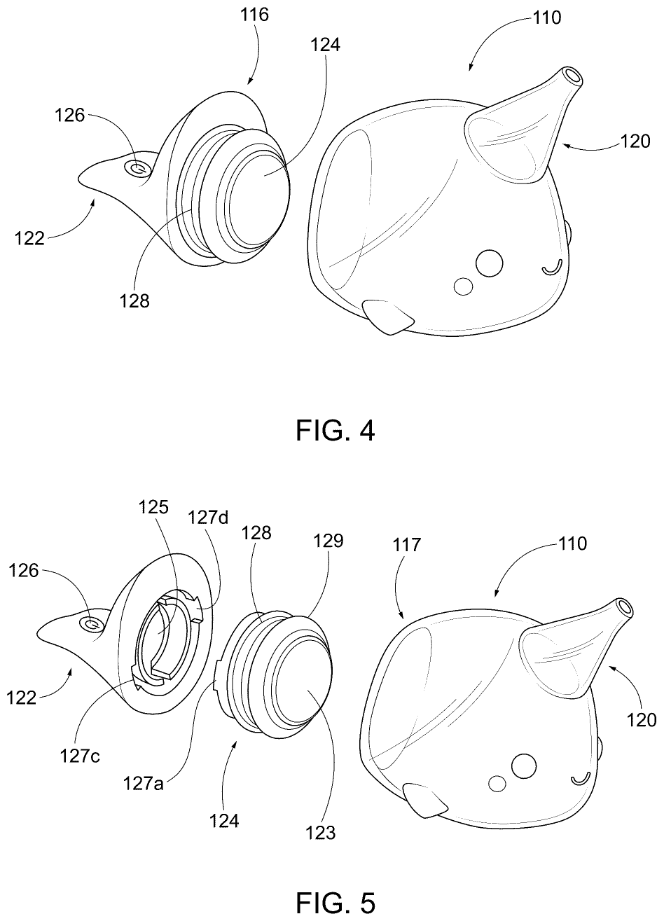

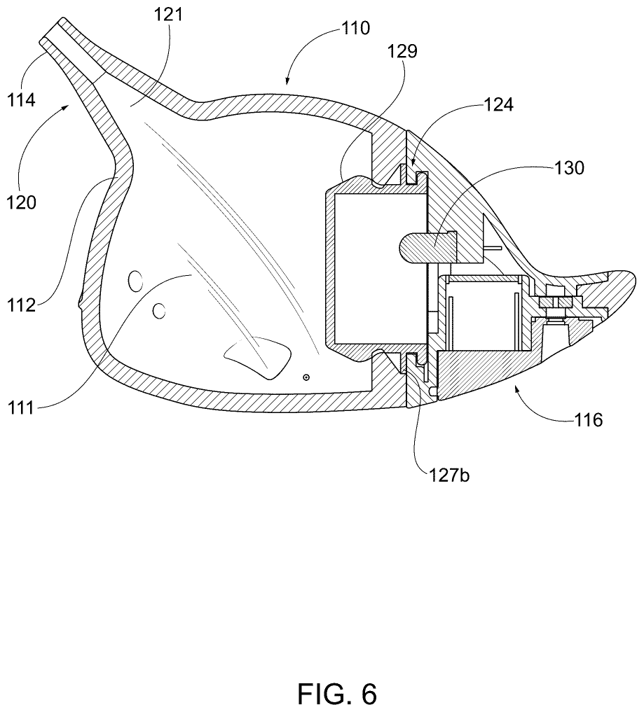

As best shown in FIG. 6, the compressible body 110 may include a rounded shape and a hollow, inner cavity 111 that is configured so that when a user squeezes the body 110 sufficient suction is created. Nasal tip 120 includes a hollow passageway 121 that extends along the length of the nasal tip 120 from a first, or insertion end 114 to a second end 112, which fluidly connects the nasal tip 120 to inner cavity 111 so that fluids can travel from the nasal tip to the inner cavity. The nasal tip may be tapered from a base or second end 112 adjacent the body 110 to an opening or insertion/distal end 114 of the tip. In use, after the insertion end 114 of the nasal tip 120 is placed adjacent or within the nostril, or other orifice, the body 110 is compressed, i.e., squeezed, to create sufficient suction to pull the mucus into passageway 121. Depending upon the viscosity and amount of mucus, or other fluid, the mucus may travel down passageway 121 and into inner cavity 111 of body 110. The body 110 may be made of any suitable material for repeated compression and at least a portion of the body 110 may be made of a translucent material. Body 110 may also be dishwasher safe for cleaning and may also be boiled in water or microwaved to clean and/or sterilize the device. In the present example silicone is utilized, although other suitable materials may also be used, as would be known to one of skill in the art. The body 110 and the housing 116 may also be detachable for ease of cleaning, for example through provision of a snap-fit, friction fit, threaded connection, or other arrangement as would be known to one of skill in the art. Referring to FIGS. 4-6, in the present embodiment, an attachment member 124 is removably detacher or attached within cavity 111 of the housing 116 by insertion of tabs 127a, 127b into corresponding slots 127c, 127d (FIGS. 5-6) and is removably detached or attached to the body 110 by flange 129. In this manner, the attachment member 124 removably secures the body 110 to the housing 116. Although the attachment member 124 in the present embodiment operates to removably secure the body 110 and the housing 116, while also providing light diffusion, it is contemplated that this may be achieved by two different pieces. For example, a light diffuser could be supported within either the body 110 or the housing 116 and the body 110 and housing 116 could be removably secured to each other by a separate attachment members such as by a male/female connection, a threaded connection, or the like. Alternatively, the body and housing may remain as a unitary device with an opening for cleaning and a light diffuser supported inside. Optionally, a removable sleeve disposed within at least a portion of the body 110 that may also be made from a soft material such as silicone, may be designed in the same overall shape of the body 110 to protect the same, and which sleeve may be removed for aiding in cleaning.

As shown in FIGS. 7 and 8, housing 116 also supports a lighting assembly 118, for example within opening 125 (shown in FIG. 5) of the housing 116. Lighting assembly 118 comprises a light 130, for example an LED bulb, a battery 132 to power the light 130, and a power button 134 to activate the light 130. A light diffusing element 123 (shown in FIG. 5) may also be provided as part of the lighting assembly 118. In the present embodiment, light diffusing element 123 is included as part of attachment member 124. For example, the light diffusing element 123 may make up at least one portion of the attachment member and be formed of a translucent material to further diffuse the light 130 to create even a softer glow than diffusion from the body 110 alone. To the extent that the attachment member 124 acts as a light diffuser, it may also be considered part of the lighting assembly 118.

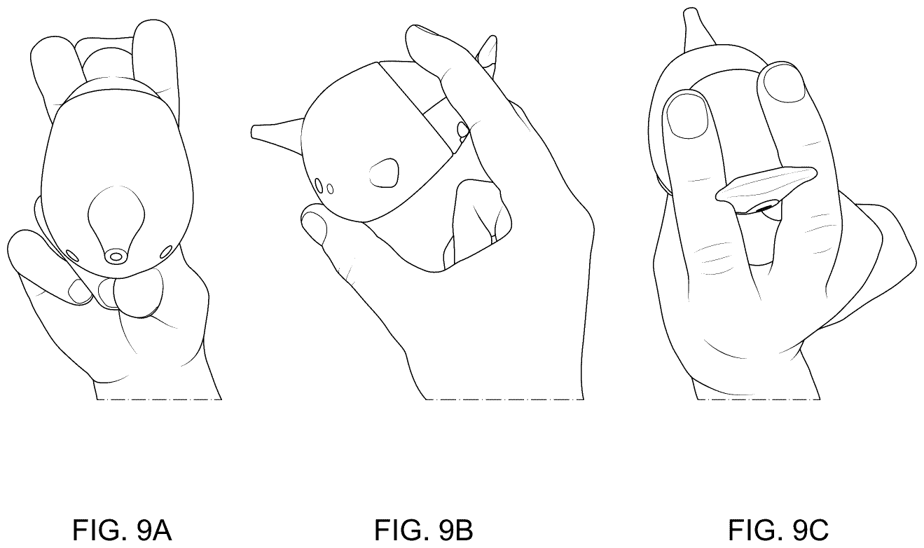

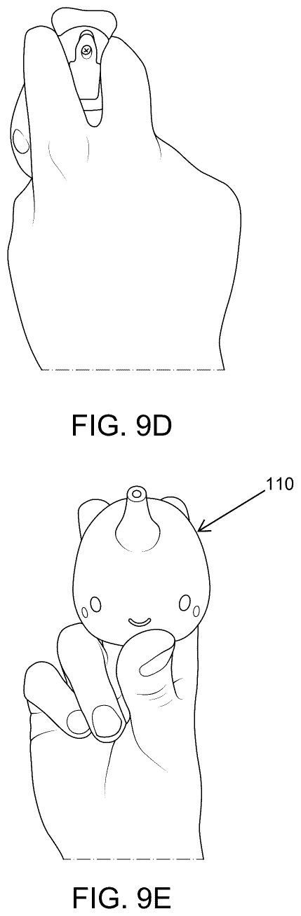

Extending proximally from the housing 116 is grip 122 that is configured to be grasped by a user during operation. Referring to FIGS. 8 and 9A-9E, grip 122 may take any of a variety of shapes that can be held by the fingers of an average adult to provide additional stability while utilizing the mucus extractor. In the present embodiment, the grip 122 has been designed in the shape of a tail to compliment the whimsical shape of the narwhal. It will be readily apparent that the grip 122 may take other shapes and styles depending upon the desired design aesthetic while being configured to be grasped by the user. The grip 122 is intended to provide stability and, therefore, is made from a more rigid material than the flexible, compressible body 110. In this respect, while the compressible body 110 changes shape as it compressed, the grip 122 retains its overall shape during use.

FIGS. 9A-9E demonstrate how the user may use the grip 122 to hold and operate the mucous remover 100. FIG. 9A illustrates a front view with the grip 122 being held between the index and middle finger while FIG. 9B shows a side view of the same grasp and FIG. 9E providing a better view of the thumb that is being used to support the body 110. FIG. 9E shows a second front view but from a slightly different angle, while FIGS. 9C and 9D show rear views of the user grasping the device. As will be appreciated, the grip 122 provides additional stability during use, allowing for one-handed use of the mucous remover.

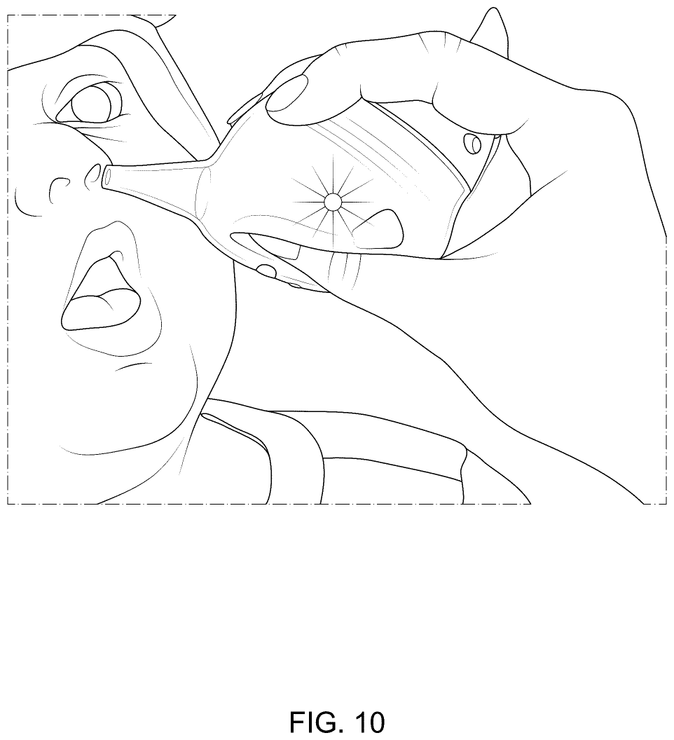

During use, the parent or other adult grasps the mucous remover device 100, for example as shown in FIGS. 6, 9A-9E and FIG. 10, places the distal end 114 adjacent the nostril or other orifice or inserts the distal end 114 of the nasal tip 120 into the nostril or other part of the baby or child's body in order to remove mucus or other fluids. Once inserted, the rounded body 110 is compressed by squeezing (as shown in FIG. 10) in order to create the suction needed to move the mucus out of the child and into the passageway 121. The mucus may then travel into the cavity 111 of body 110. If the parent is using the device in low light conditions, or simply wants an additional distraction or entertainment for the child, the light 130 may be turned on by pressing power button 134 operatively connected to the battery 132. FIG. 10 illustrates schematically the glow of the light. Turning the light 130 on will cause translucent body 110 to glow, i.e. emit or radiate dispersed lighting. The flexible body 110 is at least partially translucent and provides a soft glow by filtering the light that is emitted by the light supported in the housing when it is turned on. Alternatively, the body may be transparent.

After use, as shown in FIGS. 4 and 5, the body 110 may be separated or detached from the housing 116 by pulling to overcome the fit of the flange 129 from the body 110 and may be further disassembled by removing the attachment member 124 from the housing by a twisting motion to align the tabs 127a, 127b and corresponding slots 127c, 127d. When disassembled, the components may be washed by hand or in a dishwasher or sanitized by boiling or microwaving.

While the principles of various embodiments have been described herein, it is to be understood by those skilled in the art that this description is made only by way of example and not as a limitation as to the scope of the disclosure. Other embodiments are contemplated within the scope of the present disclosure in addition to the exemplary embodiments shown and described herein. Modifications and substitutions by one of ordinary skill in the art are considered to be within the scope of the present disclosure and the appended claims.

In addition, it is to be understood that the phraseology and terminology used herein is for the purpose of description and should not be regarded as limiting. The use of “including,” “comprising,” “containing,” “involving,” or “having,” and variations thereof herein, is meant to encompass the items listed thereafter and equivalents thereof as well as additional items while only the terms “consisting only of” or “consisting essentially of” are to be construed in a limitative sense. References to “or” may be construed as inclusive so that any terms described using “or” may indicate any of a single, more than one, and all of the described terms, as read in conjunction with the appended claims.

1. A mucus extraction device comprising:
a compressible body including an interior cavity;
a nasal tip in fluid communication with the compressible body;
a housing including a grip member, the housing extending from the body;
a light assembly disposed within at least one of the interior cavity of the body and an opening formed within the housing, the lighting assembly including a light diffusing element; and
wherein the grip member is configured to be grasped by a user during operation of the mucus extraction device and to maintain its shape when grasped.

2. The mucus extraction device of claim 1, further comprising an attachment member for removably connecting the body to the housing of the mucus extraction device.

3. The mucus extraction device of claim 2, wherein the attachment member includes the light diffusing element.

4. The mucus extraction device of claim 1, wherein the compressible body is at least partially translucent.

5. The mucus extraction device of claim 1, wherein the grip is rigid.

6. A mucus extraction device comprising:
a compressible body including an interior cavity, the body being at least partially translucent;
a nasal tip supported on a distal portion of the compressible body and in fluid communication therewith;
a housing including a grip member, the housing and grip member configured to be removably detachable from the body, the grip member being disposed at a proximal end of the mucus extraction device;
a light supported within the interior cavity of the body;
wherein the light is positioned to shine toward the nasal tip and to emit light through the at least partially translucent body.

7. The mucus extraction device of claim 6, wherein the grip member is configured to be grasped by a user during operation of the mucus extraction device.

8. The mucus extraction device of claim 7, wherein the grip member is configured to maintain its shape when grasped.

9. The mucus extraction device of claim 6, further comprising an attachment member for removably connecting the body to the housing of the mucus extraction device.

10. The mucus extraction device of claim 9, wherein the attachment member includes a light diffusing element.

11. The mucus extraction device of claim 6, further comprising a light assembly disposed within the interior cavity of the body, the lighting assembly including a light diffusing element.
